(19) United States
(12) Patent Application Publication
(10) Pub. No.: US 2010/0101954 A1
(43) Pub. Date: Apr. 29, 2010

(54) Compositions, Methods, Devices, and Systems for Nucleic Acid Fractionation

(75) Inventors: RICK CONRAD, Austin, TX (US); PATRICIA K. POWERS, Pflugerville, TX (US); LAITH VINCENT, Austin, TX (US); SCOTT HUNICKE-SMITH, Austin, TX (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/430,279

(22) Filed: Apr. 27, 2009

Related U.S. Application Data
(63) Continuation of application No. 11/373,646, filed on Mar. 10, 2006, now abandoned.
(60) Provisional application No. 60/736,438, filed on Nov. 14, 2005.

(51) Int. Cl. C07K 1/26

(52) U.S. Cl. 204/469; 204/606; 204/607; 210/497.01

(57) ABSTRACT

The present disclosure provides methods, devices, systems and compositions for nucleic acid separation and/or purification. In some embodiments, nucleic acids from about 10 nucleotides to about 150 nucleotides may be separated and/or purified in seconds to minutes. A system for purifying a nucleic acid within seconds to minutes may include: a fractionator having a housing, a first electrode, a second electrode spaced away from the first electrode, and a lower buffer chamber proximal to the second electrode; and a pre-cast gel cartridge having an upper buffer chamber and an elongate polyacrylamide gel, wherein the upper buffer chamber is in fluid communication with one end of the polyacrylamide gel, the lower buffer chamber is in fluid communication with the other end of the elongate polyacrylamide gel, the first electrode is in electrical communication with the upper buffer chamber, and the second electrode is in electrical communication with the lower buffer chamber.

### RELATED APPLICATION

This application is a continuation application of copending U.S. application Ser. No. 11/373,646 filed Mar. 10, 2006, which application claims the benefit of U.S. Provisional Patent Application Ser. No. 60/736,438, filed Nov. 14, 2005 and entitled “COMPOSITIONS, METHODS, DEVICES, AND SYSTEMS FOR NUCLEIC ACID FRACTIONATION.” The entire contents of each application are hereby incorporated in their entirety by reference.

### TECHNICAL FIELD

The present disclosure relates to methods, compositions, devices, and systems for fractionating a nucleic acid sample.

### BACKGROUND

Nucleic acids constitute a basic chemical building block of living organisms. A single nucleotide may have three component parts, namely, a base, a sugar, and a phosphate. Biologically common bases may include thymine, uracil, cytosine, adenine, and guanine. Common sugar residues include ribose and deoxyribose. Nucleotides may be linked to each other by phosphate bridges between the 3′ and 5′ positions to form linear polymers. In some cases, these polymers may be only a few nucleotides long. In others, a single molecule may include thousands or millions of nucleotides. The phosphate groups are acidic such that polynucleotides may be polyanions at normal physiological pH. Similarly, carbohydrates and proteins may include individual units (e.g., pentoses, hexoses, and amino acids), each of which may bear a charge. Thus, polynucleotides, carbohydrates, and proteins each may move according to their charge when situated in an electric field. While this may allow polynucleotides, carbohydrates, and/or proteins to be separated and/or purified, existing techniques are slow and laborious.

### SUMMARY

Accordingly, a need exists for compositions, methods, devices, and systems for more rapidly and more efficiently separating and/or purifying polynucleotides, carbohydrates, and proteins. The present disclosure provides, in some embodiments, examples of compositions, methods, devices, and/or systems for separating and/or purifying polynucleotides, carbohydrates, and proteins, e.g., on the basis of size, charge, or a ratio including both mass and charge.

For example, a sample including a polynucleotide, a carbohydrate, and/or a protein may be fractionated on a device and/or system of the disclosure to separate and/or purify one or more species of interest from other sample components.

According to some embodiments of the disclosure, a device may include a loading chamber, a sieving matrix, a collection chamber, and optionally a power source, wherein the loading chamber, the sieving matrix, and the collection chamber are in fluid communication with each other and wherein the power source, if present, is in electrical contact with the loading chamber, the sieving matrix, and the collection chamber. A loading chamber may have any geometric shape and may be configured to receive and/or contain a volume of sample and/or other material (e.g., from about fifty (50) microliters to about eleven (11) milliliters). For example, a loading chamber may be configured to receive and/or contain up to about two hundred (200) microliters, up to about four hundred (400) microliters, up to about six hundred (600) microliters, up to about eight hundred (800) microliters, and/or up to about one (1) milliliter. A sieving matrix may have any geometric shape and may be from about one (1) millimeter to about twenty (20) millimeters in each dimension. A sieving matrix may allow movement of some molecules while retarding or blocking movement of others. A collection chamber may have any geometric shape and may be configured to receive and/or contain a volume of sample and/or other material (e.g., from about fifty (50) microliters to about eleven (11) milliliters). For example, a collection chamber may be configured to receive and/or contain up to about two hundred (200) microliters, up to about four hundred (400) microliters, up to about six hundred (600) microliters, up to about eight hundred (800) microliters, and/or up to about one (1) milliliter. A collection chamber may include a species of interest during and/or after separation. A device may further include two or more electrodes, at least two of which may be in electrical communication with each other, e.g., via the loading chamber, sieving matrix, and collection chamber. A power source may be in electrical communication with the at least two electrodes.

In some embodiments, a system may include, independently, one or more of each of the following: a sample, a loading chamber, a sieving matrix, a collection chamber, a power source, a fractionation marker, and a buffer. For example, a system may include two loading chambers, two sieving matrices, two collection chambers, two loading chamber buffers, two collection chamber buffers, and one power source.

In some embodiments, a method for separating and/or purifying a polynucleotide, a carbohydrate, and/or a protein of interest may include (a) contacting a collection chamber buffer with a collection chamber wherein the collection chamber buffer is contained within at least a portion of the collection chamber, (b) contacting at least a portion of the collection chamber buffer with at least a portion of a sieving matrix, wherein the sieving matrix and the collection chamber are in fluid communication, (c) contacting at least a portion of the sieving matrix with a loading chamber, (d) contacting a loading chamber buffer with the loading chamber wherein the loading chamber buffer is contained within at least a portion of the loading chamber, (e) contacting at least a portion of the loading chamber buffer with a sample, (f) contacting at least a portion of the sieving matrix with at least a portion of the sample under conditions that permit the at least a portion of the sample to be sieved, wherein the at least a portion of the sample includes a polynucleotide, a carbohydrate, and/or a protein of interest, and (g) receiving the polynucleotide, the carbohydrate, and/or the protein of interest in at least a portion of the receiving buffer, wherein the polynucleotide, the carbohydrate, and/or the protein of interest is thereby separated and/or purified from at least a portion of at least one sample component. In some embodiments, a loading chamber, a loading chamber buffer, a sieving matrix, a collection chamber buffer, and a collection chamber may be configured and arranged to separate and/or purify a polynucleotide, a carbohydrate, and/or a protein of interest in seconds to minutes. For example, separation and/or purification may be performed in less than about twenty (20) minutes, less than about fifteen (15) minutes, less than about twelve (12) minutes, less than about ten (10) minutes, and/or less than about eight (8) minutes.

### BRIEF DESCRIPTION OF THE DRAWINGS

The patent application file contains at least one drawing executed in color. Copies of this patent or patent application publication with color drawing(s) will be provided by the United States Patent and Trademark Office upon request and payment of the necessary fee.

Some of the embodiments of the disclosure may be understood by referring in part to the following description and the accompanying drawings, wherein dimensions, unless otherwise indicated, are in inches, and wherein:

FIG. 1A shows an isometric view of a fractionator according to an example embodiment of the present disclosure in its closed position (front cover omitted);

FIG. 1B shows an isometric view of a fractionator according to an example embodiment of the present disclosure in its closed position with approximate dimensions in inches;

FIG. 2 shows a front elevation view of a fractionator Lower Housing according to an example embodiment of the present disclosure with guide lines illustrating insertion of a Lower Buffer Chamber;

FIG. 3 shows a right elevation view of a fractionator Lower Housing according to an example embodiment of the present disclosure with a section view of a portion where a Lower Buffer Chamber is inserted (guide lines);

FIG. 4 shows a right elevation view of a fractionator Upper Housing according to an example embodiment of the present disclosure with a section view of a portion where an electrode is inserted (guide line);

FIG. 5 shows an isometric view of a fractionator Upper Housing according to an example embodiment of the present disclosure;

FIG. 6 shows a front elevation view of a fractionator Upper Housing according to an example embodiment of the present disclosure;

FIG. 7 shows a left elevation view of a fractionator Upper Housing according to an example embodiment of the present disclosure;

FIG. 8 shows a section view of a fractionator Upper Housing according to an example embodiment of the present disclosure taken along lines 8-8 of FIG. 7;

FIG. 9 shows a plan view of a portion of a fractionator Upper Housing according to an example embodiment of the present disclosure;

FIG. 10 shows a plan view of a fractionator Upper Housing according to an example embodiment of the present disclosure;

FIG. 11 shows a section view of a fractionator Upper Housing according to an example embodiment of the present disclosure taken along lines 11-11 of FIG. 12;

FIG. 12 shows an upper isometric view of a connector shroud according to an example embodiment of the present disclosure;

FIG. 13 shows a lower isometric view of a connector shroud according to an example embodiment of the present disclosure;

FIG. 14 shows a left elevation view of a connector shroud according to an example embodiment of the present disclosure;

FIG. 15 shows a lower plan view of a connector shroud according to an example embodiment of the present disclosure;

FIG. 16 shows a right elevation view of a connector shroud according to an example embodiment of the present disclosure;

FIG. 17 shows an upper plan view of a connector shroud according to an example embodiment of the present disclosure;

FIG. 18 shows an isometric view of a fractionator according to an example embodiment of the present disclosure in its closed position with guide lines illustrating insertion of gold-plated pins;

FIG. 19 shows a right elevation view of a partially assembled fractionator according to an example embodiment of the present disclosure in its closed position with a section view of a portion where a gold-plated pin is inserted (guide line) and a PCA/Connector Assembly that contacts the gold-plated pin;

FIG. 20 shows a front elevation view of a translucent front cover according to an example embodiment of the present disclosure;

FIG. 21 shows a right elevation view of a translucent front cover according to an example embodiment of the present disclosure;

FIG. 22 shows a plan view of a translucent front cover according to an example embodiment of the present disclosure;

FIG. 23 shows an isometric view of a translucent top cover according to an example embodiment of the present disclosure;

FIG. 24 shows a plan view of a translucent top cover according to an example embodiment of the present disclosure;

FIG. 25 shows a left side elevation view of a translucent top cover according to an example embodiment of the present disclosure;

FIG. 26 shows a front elevation view of a translucent top cover according to an example embodiment of the present disclosure;

FIG. 27 shows a plan view of the underside of a translucent top cover according to an example embodiment of the present disclosure;

FIG. 28 shows a rear elevation view of a translucent top cover according to an example embodiment of the present disclosure;

FIG. 29 shows an isometric view of a left side cap according to an example embodiment of the present disclosure;

FIG. 30 shows a front elevation view of a left side cap according to an example embodiment of the present disclosure;

FIG. 31 shows a plan view of a left side cap according to an example embodiment of the present disclosure;

FIG. 32 shows a left elevation view of a left side cap according to an example embodiment of the present disclosure;

FIG. 33 shows a right elevation view of a left side cap according to an example embodiment of the present disclosure;

FIG. 34 shows an isometric view of a partially assembled fractionator according to an example embodiment of the present disclosure in its closed position with guide lines illustrating attachment of Upper, Front, and Lower Lenses;

FIG. 35 shows an isometric view of a partially assembled fractionator according to an example embodiment of the present disclosure in its closed position with guide lines illustrating attachment of face plate and end caps;

FIG. 36 shows a right elevation view of a fully assembled fractionator according to an example embodiment of the present disclosure in its closed position;

FIG. 37 shows an isometric view of a fractionator gel tube according to an example embodiment of the present disclosure;

FIG. 38 shows an elevation view of a fractionator gel tube according to an example embodiment of the present disclosure;

FIG. 39 shows a section view of a fractionator gel tube according to an example embodiment of the present disclosure taken along lines 39-39 of FIG. 38;

FIG. 40 shows an elevation view of a circuit board for a fractionator according to an example embodiment of the present disclosure;

FIG. 41 shows a plan view of the left side of the circuit board shown in FIG. 40;

FIG. 42 shows a plan view of the right side of the circuit board shown in FIG. 40;

FIG. 43 shows an isometric view of a fractionator in its open position with an inserted lower buffer chamber according to an example embodiment of the present disclosure;

FIG. 44 shows an isometric view of a fractionator in its open position being loaded with Lower Running Buffer according to an example embodiment of the present disclosure;

FIG. 45 shows an isometric view of a fractionator gel tube being installed in a fractionator according to an example embodiment of the present disclosure;

FIG. 46 shows an isometric view of a fractionator in its open position with an inserted fractionator gel tube being loaded with Upper Running Buffer according to an example embodiment of the present disclosure;

FIG. 47A shows a front elevation view of an assembled fractionator during a run according to an example embodiment of the present disclosure;

FIG. 47B shows an exploded view of the fractionator gel tube portion of the fractionator illustrated in FIG. 47A;

FIG. 48 shows the resolution between polynucleotide fractions collected from a fractionator according to an example embodiment of the present disclosure; and

FIG. 49 shows a 15% denaturing acrylamide gel loaded with RNA prepared by different methods and ethidium bromide-stained (upper panel) or processed for miR-16 detection (lower panel).

### DETAILED DESCRIPTION

The present disclosure relates to methods, compositions, devices, and systems for separating and/or purifying a nucleic acid, a protein, and/or a carbohydrate of interest from a sample.

In some embodiments, a sample may include at least one nucleic acid of interest, at least one protein of interest, and/or at least one carbohydrate of interest and at least one additional material. For example, a sample may include one or more isolated and/or purified nucleic acids. A sample may include a crude cell lysate. According to some embodiments, separating and/or purifying a compound of interest from a sample may include fractionating at least a portion of a sample (e.g., nucleic acids in a crude lysate) into a plurality of parts or fractions. At least a portion of a sample may be fractionated according to any physical, chemical, and/or any other feature desired. For example, molecules may be fractionated on the basis of size (e.g., molecular weight), charge (e.g., at a particular pH), and/or a mass to charge ratio. Without being limited to any particular method or means of separation and/or purification, fraction are used in the following paragraphs to illustrate some embodiments of the disclosure. Similarly, without being limited to any particular compound of interest or sample composition, nucleic acids are used in the following paragraphs to illustrate some embodiments of the disclosure.

A device according to some embodiments of the disclosure may include a fractionator. A fractionator may include, for example, at least one anode, at least one cathode, at least one loading chamber, at least one sieving matrix, and at least one collection chamber wherein the at least one loading chamber, the at least one sieving matrix, and the at least one collection chamber are in fluid communication with each other. Fluid communication may exist where solvent and/or solute molecules in one place (e.g., a loading chamber) may move to the other (e.g., a sieving matrix). According to some embodiments, a loading chamber may not be in direct fluid communication with a collection chamber. For example, fluid communication between a loading chamber and a collection chamber may be solely through a linking sieving matrix. In some embodiments, fluid communication may not exist across the solid wall(s) of a loading chamber, sieving matrix cartridge, and/or collection chamber.

In some embodiments, a device may be configured and arranged such that sieving occurs in a direction and/or along an axis substantially parallel to a gravitational vector. For example, a device in which fractionation or sieving occurs in a direction substantially parallel to the gravitational vector may include a loading chamber positioned above a sieving matrix (e.g., an upper chamber) and a collection chamber below a sieving matrix (e.g., a lower chamber). In some embodiments, a fractionator may include a housing, two electrodes spaced apart, a sieving matrix cartridge positioned between the electrodes, and a collection chamber. A sieving matrix cartridge and/or collection chamber may releasably contact each other. A sieving matrix cartridge and/or collection chamber may releasably contact at least a portion of a housing. A sieving matrix cartridge may include an upper chamber and a sieving matrix (e.g., a pre-cast gel). The dimensions of upper and lower chambers and a sieving matrix may be selected to separate and/or purify a nucleic acid in seconds to minutes. For example, separation and/or purification may be performed in less than about twenty (20) minutes, less than about fifteen (15) minutes, less than about twelve (12) minutes, less than about ten (10) minutes, and/or less than about eight (8) minutes. In some embodiments, a fractionator according to the disclosure may be used for miRNA isolation for labeling, array hybridization, and/or any other purpose.

A fractionator of the disclosure may be designed and/or optimized for visualizing and/or purifying small nucleic acids including, without limitation ribonucleic acids, deoxyribonucleic acids, and modified forms thereof. The nucleic acid may be single-, double-, and/or multi-stranded. In some embodiments, a fractionator may comprise a gel cartridge loading slot, a first electrode, and a second electrode spaced away from the first electrode. For example, a first electrode may be at or near one end of a gel cartridge loading slot and a second electrode may be near an opposing end of the gel cartridge loading slot. A fractionator may further comprise a lower buffer chamber. A lower buffer chamber may be configured to receive from about fifty (50) microliters to about eleven (11) milliliters. In addition, a fractionator according to some embodiments, may comprise a housing, a PCA/connector assembly, and/or a shroud connector. For example, a housing may include discrete units (e.g., an upper housing and a lower housing). These discrete units, in some embodiments, may be hingedly or otherwise connected to each other. Also, in some embodiments, a PCA/connector assembly may be mounted to a connector shroud. The shroud connector with attached PCA/connector assembly may be mounted to a lower housing.

In some embodiments, a fractionator of the disclosure may provide consistent separation of molecules. In some embodiments, a fractionator of the disclosure may provide predictable separation of small, single-stranded nucleic acids within seconds or minutes. In some embodiments, one may fractionate and/or purify a nucleic acid according to its time of elution, its elution relative to a molecular weight marker, and/or combinations thereof.

A fractionator of the disclosure may be configured to be conveniently placed on any laboratory bench. For example, it may be configured to have a foot-print of less than about five (5) square centimeters, less than about ten (10) square centimeters, less than about twenty (20) square centimeters, less than about thirty (30) square centimeters, less than about forty (40) square centimeters, less than about sixty (60) square centimeters, less than about eighty (80) square centimeters, less than about a hundred (100) square centimeters, and/or less than about two hundred (200) square centimeters. In some embodiments, a fractionator of the disclosure may be configured to process large samples. In some embodiments, a fractionator may be configured to process a plurality of samples in parallel. For example, a single fractionator may be configured to accommodate more than one sieving matrix. A fractionator of the disclosure, in some embodiments, may be set up before use (e.g., about 10-15 minutes).

A fractionator, according to some embodiments of the disclosure, may separate biomolecules. Without being limited to any particular mechanism of action, biomolecules may be separated, for example, using an electromotive force to drive desired molecules through a sieving matrix that retards, restricts, or blocks larger, unwanted molecules from passage.

A loading chamber (e.g., an upper chamber), a sieving matrix, and/or a collection chamber may be configured to accommodate any mass of nucleic acids. For example, a fractionator gel may be configured to be loaded with more than about one hundred micrograms of total nucleic acid (e.g., about one milligram). Alternatively, a fractionator gel may be configured to be loaded with less than about one hundred micrograms of total nucleic acid (e.g., about one hundred nanograms). In some specific example embodiments, approximately 10 ng of small RNA may be recovered from a 100 μg total RNA sample. In addition, according to some embodiments, a substantial fraction of the nucleic acids above a pre-selected molecular weight are excluded. For example, where the pre-selected molecular weight cut off is forty (40) nucleotides, methods, devices, and systems of the disclosure may be configured to exclude most (e.g., more than about 70%, more than about 75%, more than about 80%, more than about 85%, more than about 90%, more than about 95%, more than about 98%, more than about 99%, or more) of the nucleic acids with a higher molecular weight.

Resolution, in some embodiments, may be influenced by, for example, pH, gel pore size, gel length, and/or current. A matrix may, for example, separate molecules on the basis of charge and/or apparent size. Charge may be affected by the pH (increasing positive charge at low pH, increasing negative charge at high pH) to increase or decrease charge-to-size ratio. Apparent size may also be influenced by the presence of denaturants such as urea, which may tend to unfold proteins and/or loosen the binding between nucleic acid duplexes. Different ranges of sizes may be separated by careful design of the matrix, so that the average pore size of the matrix allows relatively rapid migration of the species of interest while impeding those molecules of undesired size and/or charge. The length of the matrix may manipulated as well to achieve a balance between speed (short length) and resolution (longer lengths). The sieving time may also be reduced by sufficiently increasing current to heat the matrix without without damaging it or the sample.

A sieving matrix, according to some embodiments of the disclosure, may be configured to accommodate a nucleic acid (e.g., RNA) load of up to about one (1) milligram. In other embodiments, a sieving matrix may be configured to accommodate a nucleic acid (e.g., RNA) load of from about 1 μg to about 100 μg.

In some embodiments, a sieving matrix may include a gel, a membrane, a gel filtration column, a cross-linked plastic, a fused frit, and the like. A sieving matrix may be homogeneous in some embodiments. For example, a sieving matrix may include a polyacrylamide gel with uniform pore sizes along its length. A sieving matrix may not be homogeneous. According to some embodiments, a sieving matrix may include a membrane and the instrument may be used for electro-elution of charged species from porous samples. A fractionator, in some embodiments, may include a polyacrylamide gel sieving matrix, but the same process may be applied to any potential matrix with pore sizes small enough to separate the charged molecules in question.

In some embodiments, a sieving matrix cartridge may include a sieving matrix and a sieving matrix wall. For example, a sieving matrix may be at least partially enclosed by a sieving matrix wall. A sieving matrix wall may have a generally cylindrical shape (or any other geometric shape) with openings on opposing ends. A sieving matrix wall may be include any non-conducting or substantially non-conduction material. A sieving matrix wall may be configured to contact a loading chamber and/or a collection chamber according to some embodiments. For example, a sieving matrix wall may be configured to releasably or permanently contact a loading chamber. A loading chamber and a sieving matrix cartridge may form a single, contiguous unit. A sieving matrix cartridge may be configured to be reusable and/or disposable. For example, a new sieving matrix cartridge may be installed in a fractionator prior to every sample loading and/or fractionation and/or removed after every fractionation.

In some embodiments, a sieving matrix and a sieving matrix wall may be formed separately or together. For example, a polyacrylamide gel may be cast and subsequently installed in or otherwise surrounded by a sieving matrix wall. Alternatively, a polyacrylamide gel may be cast within a sieving matrix wall (e.g., tube). In some embodiments, a sieving matrix wall may be contacted with another component of a fractionator (e.g., a collection chamber) prior to formation and/or placement of a sieving matrix. In some embodiments, a sieving matrix may be prepared in advance of when it is assembled into a fractionator (e.g., “pre-cast). For example, a matrix may be set into a impermeable “shell” or “cassette” that may then be placed in a fractionator with loading and/or collection chambers integrated into it.

A sieving matrix may have any geometrical shape including, without limitation, a cylinder, a cube, and a toroid. According to some embodiments, a sieving matrix (e.g., a polyacrylamide gel) may be generally cylindrical with a diameter of from about one (1) millimeter to about twenty (20) millimeters and a length along its longitudinal axis of from about one (1) millimeter to about two hundred (200) millimeters. In a specific example, a polyacrylamide gel may be from about five (5) millimeters to about twenty (20) millimeters long. In another specific example, a polyacrylamide gel may be 0.25 inches wide by 0.56 inches long. According to some embodiments, an electrostatic force may be exerted, for example, across, through, and/or along the length of a sieving matrix.

A sieving matrix, according to some embodiments, may include polyacrylamide at a concentration of from about 4% to about 20% (v/v) with acrylamide:bisacrylamide ratios of from about 10:1 to about 100:1. For example, a polyacrylamide concentration may be from about 4% (v/v) to about 8% (v/v), from about 8% (v/v) to about 12% (v/v), from about 12% (v/v) to about 16% (v/v), from about 16% (v/v) to about 20% (v/v), about 8% (v/v) to about 10% (v/v), from about 9% (v/v) to about 11% (v/v), from about 10% (v/v) to about 12% (v/v), from about 8% (v/v) to about 11% (v/v), from about 9% (v/v) to about 12% (v/v), and/or from about 4% (v/v) to about 12% (v/v). Similarly, an acrylamide:bisacrylamide ratio may be from about 10:1 to about 25:1, from about 10:1 to about 15:1, from about 15:1 to about 20:1, from about 20:1 to about 25:1, from about 25:1 to about 50:1, from about 50:1 to about 75:1, and/or from about 75:1 to about 100:1. In a specific example embodiment, a sieving matrix may include polyacrylamide at a concentration of about 10% (v/v) with at an acrylamide:bisacrylamide ratio of about 14:1.

A fractionator system of the disclosure may be configured, according to some embodiments, for separation and/or purification of nucleic acids—with gel run times of about 10-12 minutes. According to some embodiments, a fractionator system may be configured to be similar to one-dimensional electrophoresis except that small RNAs may be run through the entire gel rather than simply into it. For example, a fractionator system of the disclosure may pass one or more nucleic acids through a denaturing gel matrix, e.g., into a lower buffer collection chamber. A lower buffer chamber may be designed for retention of eluted material, for example, to facilitate further nucleic acid purification. This may be accomplished by using a very short gel length with an optimized gel composition. In some embodiments, this gel may allow very small RNA species, in the range of up to 40 nucleotides, to pass through the gel in about ten to twelve minutes, leaving larger species trapped in the gel matrix.

A fractionator according to some embodiments of the disclosure may be configured to accommodate very small volumes in the upper and lower electrode buffer chambers. For example, a lower chamber may only hold about 0.25 mL of solution, so eluted nucleotides in this volume may be easily used in a precipitation with ethanol (adding 875 μL of ethanol may be sufficient to precipitate small RNA).

A fractionator system of the disclosure may comprise a fractionator of the disclosure, a matrix (e.g., a pre-cast sieving matrix cartridge), and/or one or more aqueous buffers. A fractionator system of the disclosure may further comprise a current source, a safety cut-off switch, an in-operation signal, and/or a molecular weight marker. A current source may be selected from the group consisting of an alternating current (e.g., a wall outlet) and direct current (e.g., a battery). For example, a system according to some embodiments, may comprise a power-cord containing standard plugs at one end-thus permitting use with most common laboratory power supplies. In some embodiments, a fractionator may have a unit-to-unit power connection. Unit-to-unit power connections may be configured to run a plurality of fractionators in parallel from a single gel power supply.

Each component of a fractionator system of the disclosure may be configured to perform consistently and/or reliably from experiment to experiment. This may be facilitated, in part, by manufacturing each system component (e.g., each instrument, gel, buffer, and/or reagent) in compliance with strict ISO 9001 requirements.

According to some embodiments, an upper chamber buffer and a lower chamber buffer may be used. The volume of a buffer used in any particular embodiment may require optimization within the skill of those of ordinary skill in the art. Since a small volume may polarize more quickly, the buffering capacity of an upper chamber buffer may need to be increased. However, increasing the buffering capacity may interfere with adsorption on glass filter fibers. Indeed, this may become irreversible with high ethylene diamine tetra-acetic acid (EDTA). In some embodiments, an upper chamber buffer may comprise Tris(hydroxymethyl)aminomethane (“Tris”) and boric acid at a molar ratio of from about 2:1 (Tris:borate) to about 1:1. An upper running buffer may further comprise a non-ionic detergent. A non-ionic detergent may increase the ease of recovery of the solution and may inhibit RNA sticking to the sides of the buffer chamber. In addition, an upper chamber buffer may, in some embodiments, be substantially free of EDTA. According to a specific example embodiment, an upper running buffer may comprise 0.45 M Tris, 0.45 M Boric acid, and 0.2% (v/v) Triton X-100 (octylphenol ethoxylate).

An upper chamber buffer may or may not be identical to a lower chamber buffer. For example, the pH, pI, and/or composition of the two may be independently the same or different. In some embodiments of the disclosure, the pH and/or pI of the upper chamber buffer and/or the lower chamber buffer may be any pH suitable for molecular (e.g., nucleic acid) visualization, purification, and/or isolation. An upper and/or lower chamber buffer independently may have a pH that is, for example, between about 6.0 and about 9.0. An upper and/or lower chamber buffer independently may have a pH between about 7.5 and about 8.8. An upper and/or lower chamber buffer independently may have a pH between about 8.0 and about 8.3. An upper and/or lower chamber buffer independently may have a pH above or equal to 8.0. An upper and/or lower chamber buffer independently may have a pH below 8.0. In some embodiments, an upper chamber buffer may comprise tris-borate-EDTA (TBE), e.g., about 90 mM tris-borate and about 1 mM EDTA, and have a pH between about 6 and about 9. In some embodiments, an upper and/or lower chamber buffer independently may comprise tris, tricine, and/or bicine.

An upper chamber buffer, in some embodiments, may comprise a molecular weight marker. Non-limiting examples of molecular weight markers that may be used include Xylene Cyanol, Acid Violet 17, Alkali Blue 6B, Alphazurine, Eosin Y, Guinea Green, Lissamine Green B, Sulforhodamine B, and/or Violamine R. In addition, molecules with a lower charge-to-mass ratio may be used to mark larger RNA species since they may be expected to migrate more slowly. A molecular weight marker may be selected to elute at the same molecular weight as a nucleic acid of interest under normal running conditions to serve as a reference point for elution of the nucleic acid of interest. For example, a molecular weight marker may be selected to elute at the same molecular weight as a single-stranded RNA molecule of 40 bases.

A fractionator system of the disclosure may be used in connection with a fast, easy, and/or convenient method for purification of small nucleic acids. In some specific example embodiments, nucleic acids may be purified by polyacrylamide gel electrophoresis (PAGE).

A streamlined procedure, according to some embodiments, may comprise fractionation of a sample comprising at least one nucleic acid, carbohydrate, and/or protein. For example, nucleic acid may be fractionated by a sieving matrix cartridge. The gel loading capacity of the molecule to be separated generally may be determined empirically and may depend, in part, upon the number of molecules of interest to be separated (e.g., concentration), the size of the molecule(s) of interest, the mass-to-charge ratio of the molecule(s) of interest, the size of other molecules in the sample, the number of other molecules in the sample, the nature of the sieving matrix, and/or the porosity of the sieving matrix. In some embodiments, the gel loading capacity for nucleic acids is more than from about one (1) micrograms to about one hundred (100) micrograms.

Molecules running through a gel may be deposited in a lower collection chamber. With this system, one may rapidly purify molecules (e.g., nucleic acids) under a selected molecular weight cut-off (e.g., 40 bases) by terminating electrophoresis at the time a molecular weight marker elutes from a gel. The lower portion of the chamber may be emptied at any point during a run. For example, in some embodiments, substantially all nucleic acids with a molecular weight of about 20 bases and about half of all nucleic acids with a molecular weight of about 40 bases may be collected by stopping a run when a 40-base marker elutes from a pre-cast gel. The run may be continued, in some embodiments, to deposit the balance of about 40-base nucleic acids in the lower chamber. Continuing the run may result in elution of some higher molecular weight nucleic acids, in some instances.

In some embodiments, serial collection of lower buffer may be performed to obtain several size populations from the master sample. For example, aliquots of up to the entire volume of a collection chamber buffer may be removed and the collection chamber may be replenished with fresh collection chamber buffer from time to time during sieving. Each aliquot may have a distinct population of molecules. At longer times, nucleic acid species (e.g., RNA) up to or even over 150 nucleotides may be isolated. With proper selection of buffers and gel composition (e.g., a lower acrylamide concentration and/or lower acrylamide to bisacrylamide ratio), nucleic acids from about ten (10) nucleotides to about two thousand (2000) nucleotides one hundred size may be separated.

A method for purifying and/or fractionating nucleic acid may, in some embodiments, include pipetting an aliquot of lower running buffer into a lower buffer chamber, inserting a pre-cast fractionator gel, pipetting an aliquot of upper running buffer into an upper buffer chamber, adding a nucleic acid sample (e.g., about 100 μg), applying a constant voltage for a discrete period of time or until a molecular weight marker reaches a defined point (e.g., lower buffer chamber).

In some specific example embodiments, a method of the disclosure may include:
Pipetting 250 μL of lower running buffer into a lower buffer chamber;
Inserting a pre-cast fractionator gel cartridge into a fractionator of the disclosure;
Pipetting 250 μL of upper running buffer into an upper buffer chamber;
Adding a sample nucleic acid (up to 100 μg of nucleic acid) in, for example, water or a low ionic strength buffer;
Applying a potential (e.g., a constant voltage of about 75-80 V and about 2-5 mA for a single fractionator) for approximately 10 to 14 minutes and/or until a molecular weight marker (e.g., blue dye) begins to exit the gel; and
Collecting a separated and/or purified nucleic acid from lower buffer chamber.

A method of the disclosure may further comprise, in some embodiments, pre-purification and/or isolation of small nucleic acids including, without limitation, small ribosomal RNA (5S rRNA), transfer RNA (tRNA), microRNA (miRNA), or small interfering RNA (siRNA). For example, a relatively crude biological extract comprising nucleic acids may be subjected to organic extraction followed by purification on a glass fiber filter to isolate total RNA ranging in size from kilobases down to 10-mers.

The resulting nucleic acid may be purified or concentrated further. For example, an organic extraction and glass fiber filter method may be used, e.g., when from about 2 μg to about 100 μg nucleic acid was loaded onto a fractionator of the disclosure. When less than 2 μg nucleic acid was loaded onto the fractionator, overnight sodium acetate/ethanol precipitation may be used for recovery of the <40 nucleotide fraction from the lower running buffer. Note that for quantitative recovery of small nucleic acids, the precipitation may be incubated overnight at −20° C.

In some embodiments, nucleic acids eluted from a fractionator of the disclosure may be subjected to solid phase extraction on glass fiber filters (GFF) that may then be eluted in about or over 10 μL of low-salt solution. This may have an added benefit of removing free nucleotides and oligonucleotides smaller than about n=10.

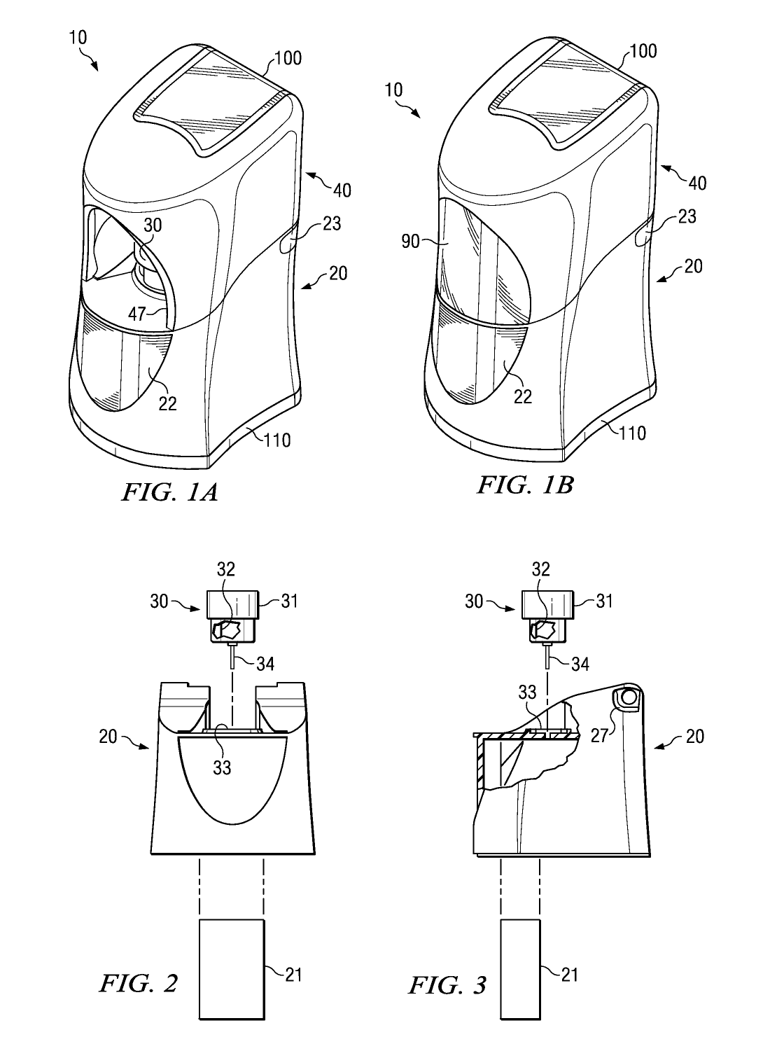

FIGS. 1A and 1B show an example fractionator 10 in its closed position. Fractionator 10 may include lower fractionator housing 20 and upper fractionator housing 40, which may be hingedly connected to each other. lower fractionator housing 20 may include face plate 22, end cap 23, lower buffer chamber 30, and base 110. Base 110 may be frosted and/or tinted. Upper fractionator housing 40 may include viewing aperture 47, front translucent cover 90, and top translucent cover 100.

FIGS. 2 and 3 show of an example lower fractionator housing 20 with guide lines illustrating insertion of lower buffer chamber 30 and optional counterweight 21. Lower buffer chamber 30 insertion may include directly or indirectly electrically contacting wire 34 to LED 113 and/or a terminal. Inserted lower buffer chamber 30 may contact lower fractionator housing 20 (e.g., releasably, rotatably, fixedly, and otherwise) at or near lower chamber cutout 33. Counterweight 21 may contact lower fractionator housing 20 (e.g., releasably, rotatably, fixedly, and otherwise). Lower buffer chamber 30 may include lower buffer chamber wall 31, lower buffer chamber gel tube aperture 32, and wire 34. Lower buffer chamber wall 31 may encircle and/or define lower buffer chamber gel tube aperture 32. Lower buffer chamber wall 31 may be configured to either alone or in combination with lower buffer chamber cutout 33 define a volume that may sealably contain a liquid. Wire 34 may be in electrical contact with at least a portion of the volume.

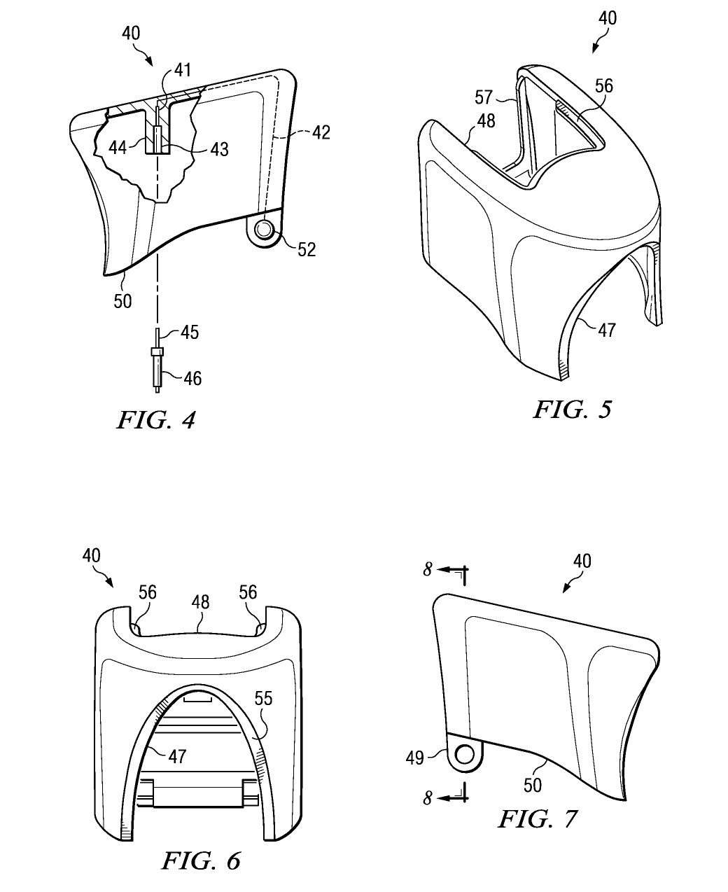

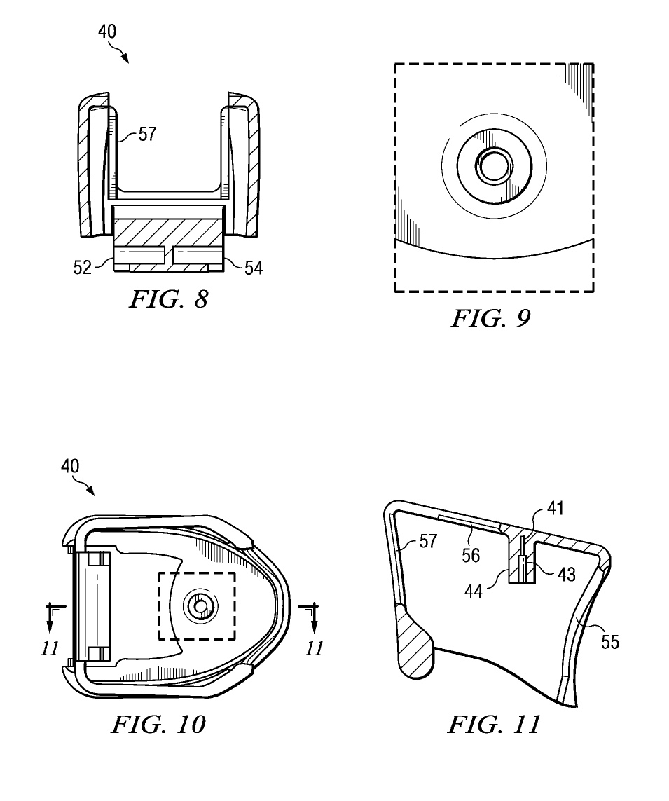

FIGS. 4-11 show an example upper fractionator housing 40, which may include wire 41, wire housing 43, wire 42, upper chamber electrode cutout 44, wire 45, and wire housing 46. Wire housing 43 may enclose at least a portion of wire 41. Wire housing 46 may enclose at least a portion of wire 45. Wire 42 may extend from about hinge 49 to about upper chamber electrode cutout 44 and my be in electrical contact with wire 41. Wire 42 may electrically contact conductive pin 51 and/or conductive pin 53. Wire 45 and wire housing 46 may be inserted into lower chamber housing 40 such that wire 45 is in electrical contact with wire 41 (FIG. 4, guide lines). At least a portion of the lower edge or surface of fractionator upper housing 40 may define upper housing contact surface 50 which contacts at least a portion of fractionator lower housing 20. Fractionator upper housing 40 may further include conductive pin recess 52, conductive pin recess 54, cover mounting surface (front) 55, cover mounting surface (top) 56, and cover mounting surface (back) 57 (FIGS. 5, 6, and 8). Mounting surfaces along the edges of aperture 48 may be positioned and configured to contact and/or support a cover. FIGS. 9 and 10 show a protuberance that may house an upper electrode in an upper fractionator housing 40. FIG. 11 shows wire 41, wire housing 43, and upper chamber electrode cutout 44.

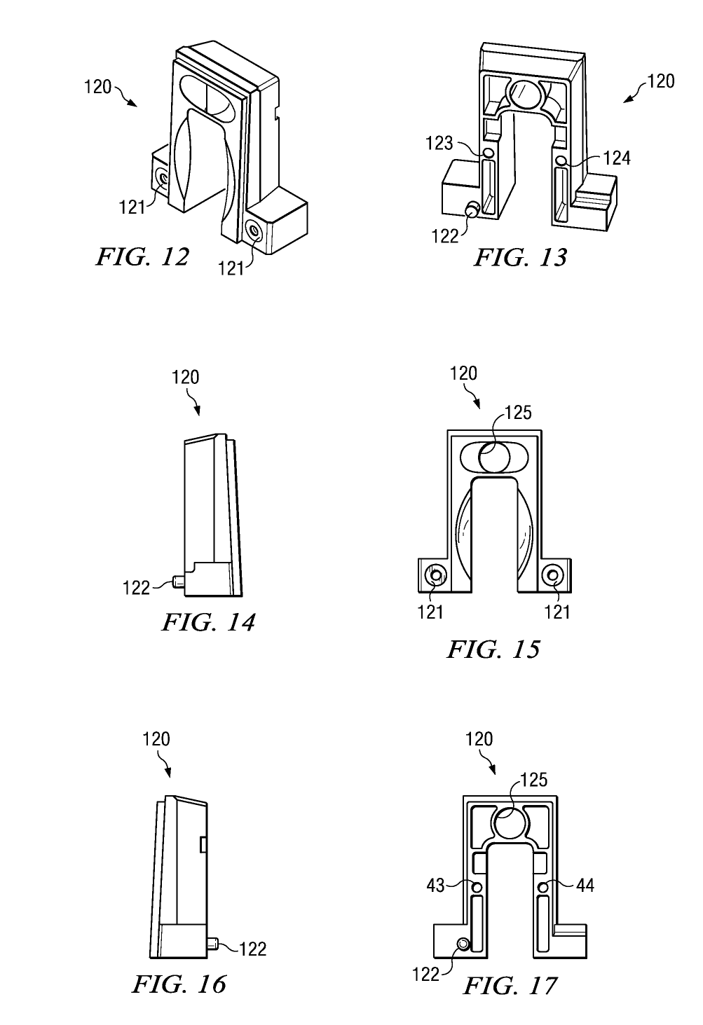

FIGS. 12-17 show an example connector shroud 120, which may have a shape loosely similar to an inverted “U” or a horseshoe and may include brass fitting 121, locator 122, anchor hole 123, anchor hole 124, and aperture 125. Brass fitting 121 may be made of brass or other material and may be configured (e.g., threaded) to receive a connector (e.g., screw). Brass fittings 121 may be fixedly mounted to connector shroud at or near the ends of the arms of the “U” (FIGS. 12 and 15). Locator 122 may be fixedly attached to and extend above the body of connector shroud 120 (FIGS. 14 and 16). Locator 122 may be located on the surface opposite the surface with brass fittings 121. The surface of connector shroud 120 having locator 122 may also include anchor hole 123 and anchor hole 124 positioned at or near the middle of each opposing arm of the “U” (FIGS. 13 and 17). Anchor hole 123 and anchor hole 124 each may be configured and arranged to receive a connector on circuit board 130. Connector shroud 120 may include aperture 125 at or near the middle or apex of the inverted “U” (FIGS. 15 and 17). Aperture 125 may extend up to completely through the thickness of connector shroud 120.

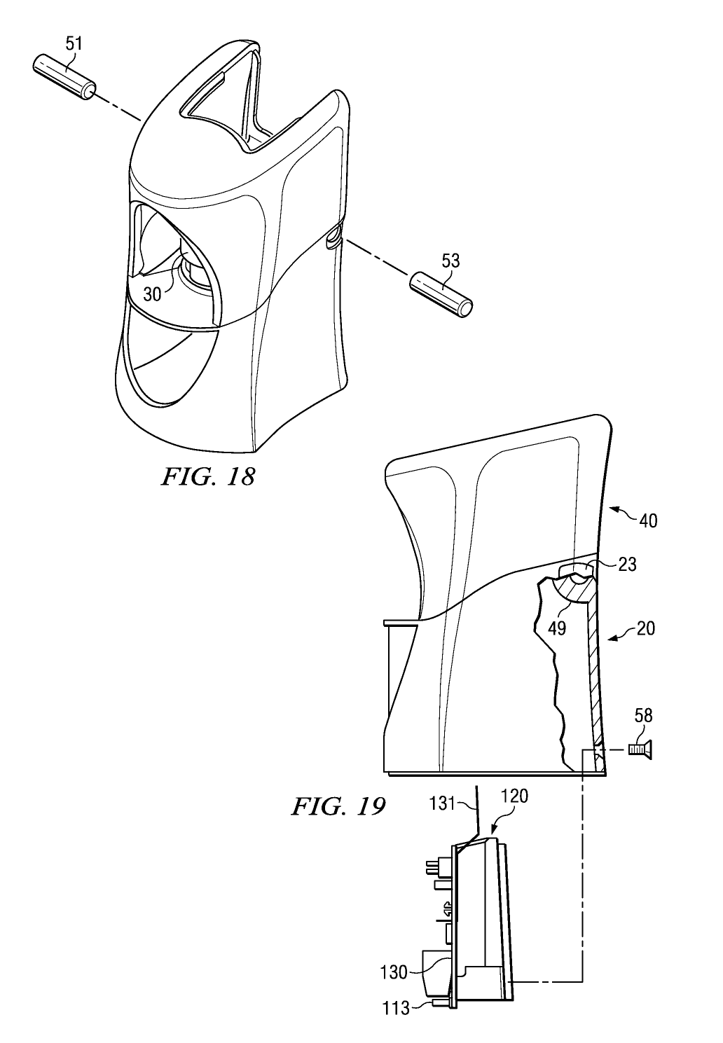

FIG. 18 shows an example fractionator 10 in a state of partial assembly with guide lines illustrating insertion of conductor pin 51 and conductor pin 53 into conductor pin recesses (not expressly shown). Conductor pins 51 and 53 may be made of and/or coated with any electrically conductive material (e.g., gold).

FIG. 19 shows an example fractionator 10 in a state of partial assembly with guide lines illustrating insertion of a connector shroud 120—circuit board 130 assembly and connector shroud anchor screw 58 (FIG. 19). Connector shroud anchor screw 58 may be made of and/or coated with any electrically conductive material (e.g., gold).

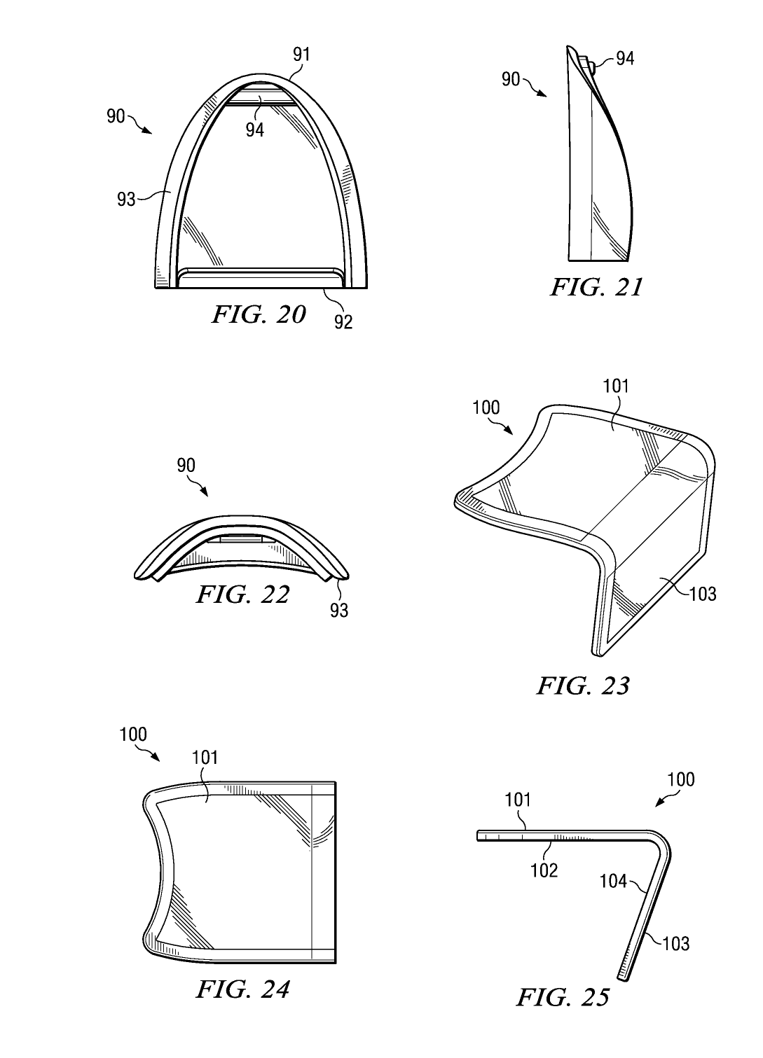

FIGS. 20-22 show an example front cover 90. Front cover 90 may be made of any material that provides an adequate barrier to exogenous materials and/or adequate structural support. At least a portion of front cover 90 may be made of a material that affords an operator a view of, for example, lower buffer chamber 30 and/or other nearby components. For example, front cover 90 may be diaphanous, transparent, and/or translucent. Front cover 90 may also be configured to enhance and/or alter (e.g., magnify) the view of the fractionator interior. The edges of front cover 90 may be defined by an arched and/or inverted U-shaped upper edge 91 and a transverse lower edge 92 (FIG. 20). At least a portion of a mounting surface 93 may abut or be near upper edge 91. In addition, an upper rib 94 may abut or be near the apex or middle of upper edge 91. Front cover 90 and its edges 91 and 92 may be configured and arranged to be complimentary to the edges of viewing aperture 47 (FIGS. 1A, 21 and 22).

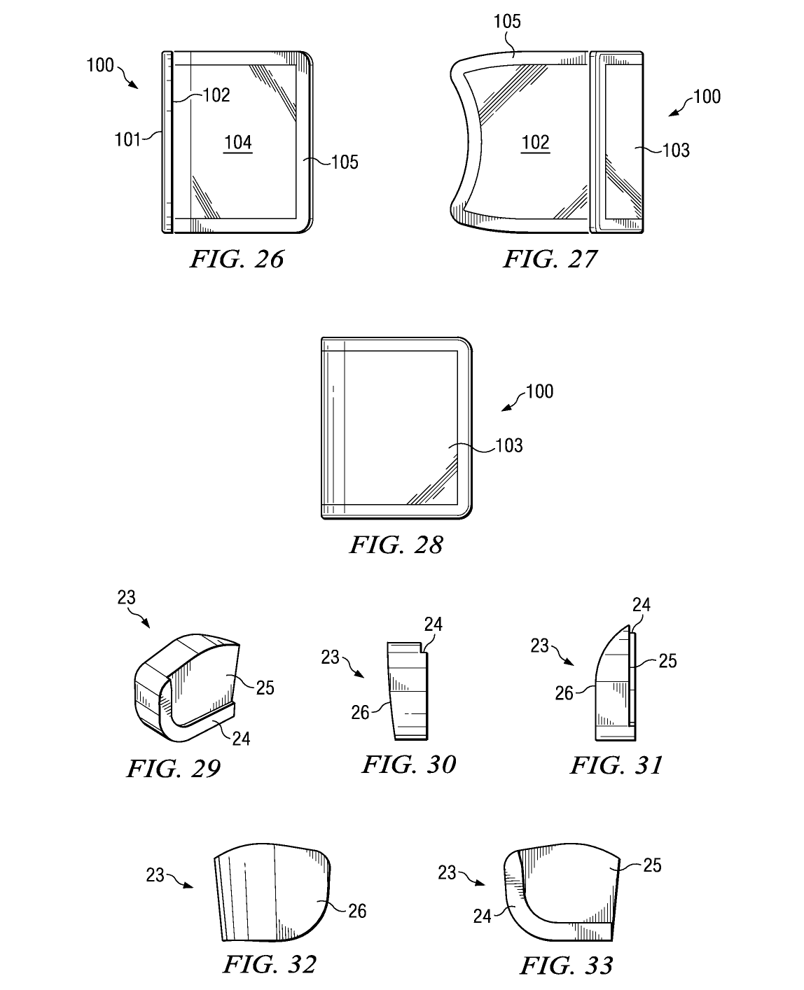

FIGS. 23-28 show an example top cover 100. Top cover 100 may be made of any material that provides an adequate barrier to exogenous materials and/or adequate structural support. At least a portion of top cover 100 may be made of a material that affords an operator a view of, for example, upper buffer chamber 80 and/or other nearby components. For example, top cover 100 may be diaphanous, transparent, and/or translucent. Top cover 100 may also be configured to enhance and/or alter (e.g., magnify) the view of the fractionator interior. Top cover 100 may be configured and arranged to be complimentary to the edges of aperture 48 (FIGS. 5, 6 and 23) and/or contact at least on of mounting surfaces 55, 56, and 57. Top cover 100 may be configured and arranged to resemble a flat, approximately rectangular sheet bent along a line that is perpendicular to longest axis in the plane of the sheet. Thus, top cover 100 may include upper surface 101, inner upper surface 102, lateral surface 103, inner lateral surface 104, mounting surface 105 (FIGS. 25 and 26).

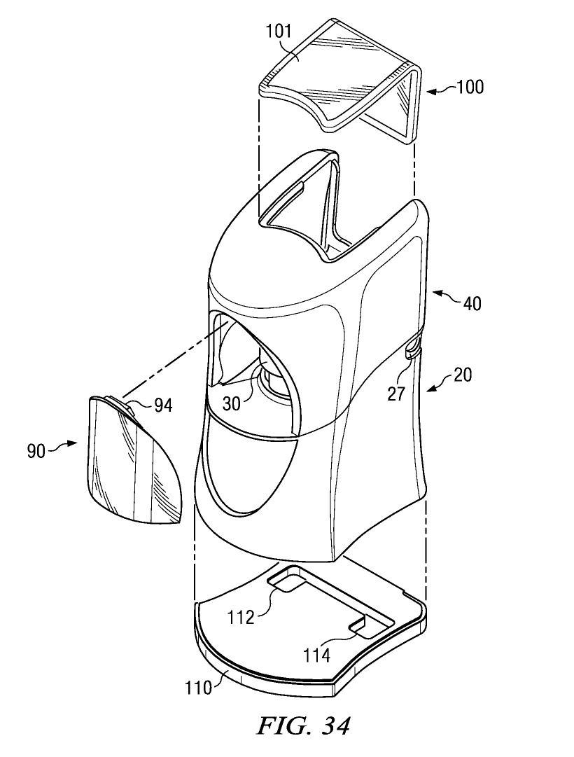

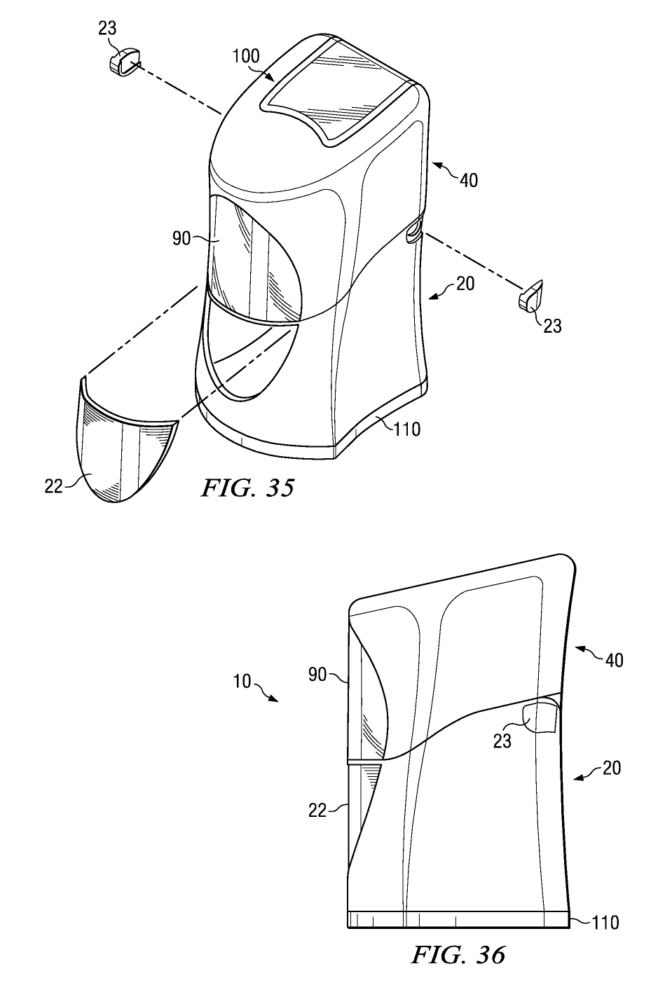

FIGS. 29-33 show an example end cap 23, which may be made of any non-conductive material. End cap 23 may be configured for releasable or fixed attachment to lower fractionator housing 20 and/or upper fractionator housing 40 at or near hinge 49 (FIG. 35, guide lines). End cap 23 may include end cap locator ridge 24, end cap inner surface 25, end cap outer surface 26, and end cap locator detent 27. Once in its finished position, end cap 23 may cover at least a portion of conductive pin 52 or 54 and form an approximately smooth, uniform lower fractionator housing 40 external surface (FIGS. 1A and 1B).

FIG. 34 shows an example fractionator 10 in a state of partial assembly with guide lines illustrating attachment of front cover 90, top cover 100, and base 110. Optional base 110 may be frosted and/or tinted and may include LED cutout 112 and LED cutout 114. Base 110 and its cutouts may be configured and arranges such that upon activation of LED 111 and/or LED 113, base 110 is illuminated.

FIG. 35 shows an example fractionator 10 in a state of partial assembly with guide lines illustrating attachment of face plate 22 and end caps 23. Optional face plate 22 may include space for stamping, printing, or otherwise recording information (e.g., about the fractionator, gel, buffers, sample, and/or combinations thereof). Alternatively, at least a portion of face plate 22 (and the relevant underlying portion of lower fractionator housing 20) may be made of a material that affords an operator a view of, for example, lower buffer chamber 30 and/or other nearby components.

FIG. 36 shows an assembled, example fractionator 10 in its closed position.

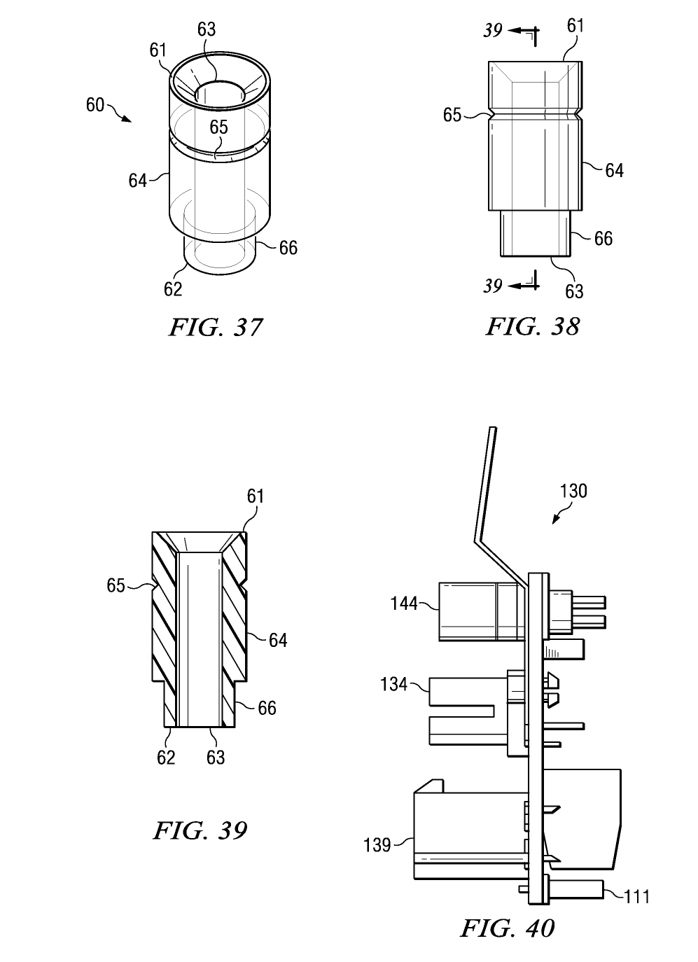

FIGS. 37-39 show an example gel tube 60. As shown, gel tube 60 optionally may have a shape that approximates a hollow cylinder defined by gel tube wall 64. Gel tube wall 64 may define gel aperture 63 and may include a gel tube upper end 61 and a gel tube lower end 62. Gel tube wall 64 may be up to completely encircled on its exterior wall by gel tube wall detent 65. Gel tube 60 may include a gel tube-lower chamber fitting 66 that may be configured and arranged to releasably contact lower buffer chamber gel tube aperture 32 and support a contiguous liquid and/or electrical connection between at least a portion of gel tube aperture 63 and at least a portion of lower buffer chamber 30. Up to the entire volume of gel aperture 63 may be occupied by gel 70. Upper buffer chamber 80 may include any portion of gel aperture 63 above gel 70 and the volume defined by the upper end 61, gel tube wall 64, and/or a plane at the uppermost edge of gel aperture 63.

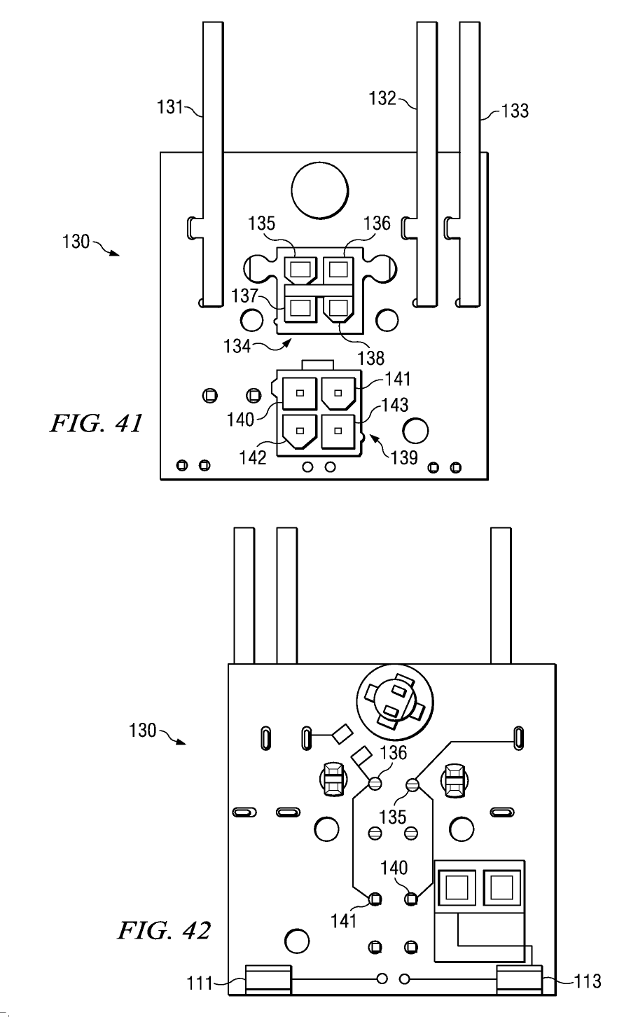

FIGS. 40-42 show an example circuit board 130, to which may be mounted spring contacts 131, 132, and 133. Each spring contact independently may protrude over an edge of circuit board 130 such that the protruding end may electrically contact, for example, conductive pin 51 and/or conductive pin 53. This contact may be under tension. Each spring contact may formed to include conductive and/or resilient materials. Each spring contact 131 independently may be in electrical contact with fuse 144, one or more terminals of power input connector 134, one or more terminals of unit output connector 139, LED 111, and/or LED 113.

### EXAMPLES

Some specific embodiments of the disclosure may be understood, in part, by referring, at least in part, to the following examples. These examples are not intended to represent all aspects of the disclosure in its entirety. Variations will be apparent to one skilled in the art.

### Example 1

### Fractionator Set Up

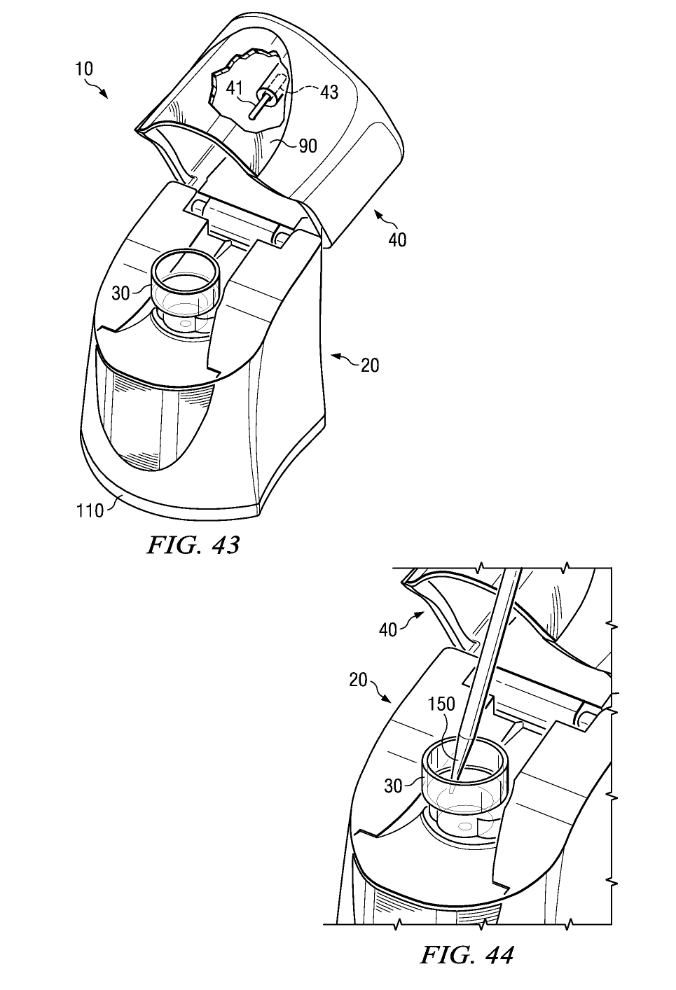

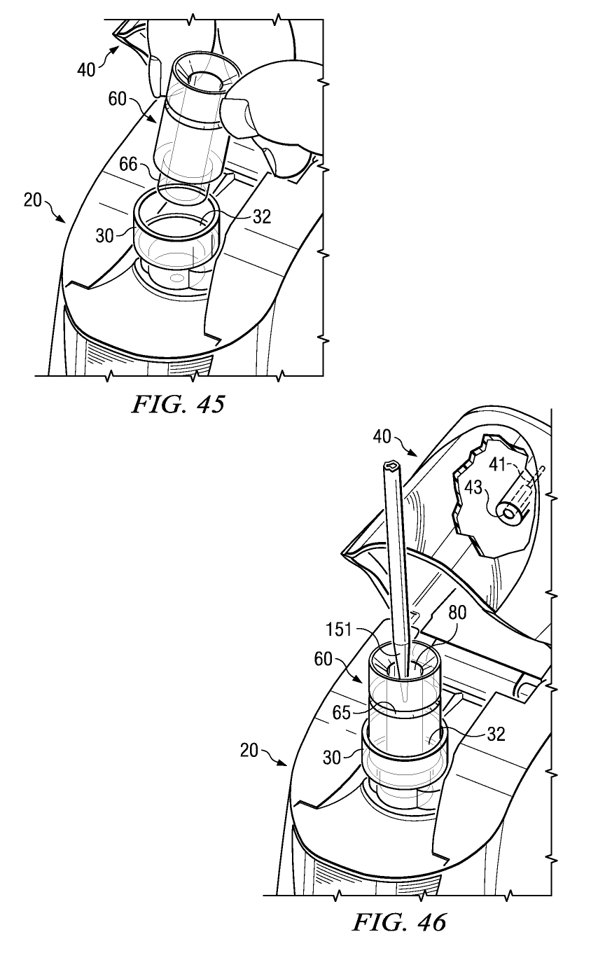

To set up a fractionator, the upper fractionator housing was lifted and swung backwards relative to the lower fractionator housing (FIG. 43). Next, 250 μL of lower running buffer was placed in a lower buffer chamber using pipet tip 150 (FIG. 44) and a pre-cast fractionator gel 60 was inserted (FIG. 45). Next, 250 μL of upper running buffer was placed in an upper buffer chamber 80 (FIG. 46).

### Example 2

### Sample Preparation and Loading

Equal volumes of (a) either a composition comprising RNA or a composition comprising ssDNA and (b) a fractionator loading buffer comprising A40, a molecular weight marker, were mixed such that the total volume was under 100 μL. The mixture was heated to 95° C. for 2 minutes to denature the nucleic acid and then placed on ice until it was loaded onto the upper surface of a pre-cast gel. Once the sample was loaded, the fractionator was closed. The upper electrode should contact upper running buffer. If necessary, more upper running buffer may be added to achieve contact.

### Example 3

### Fractionator Operation

A fractionator of the disclosure configured to be connected to an electrophoresis power source was connected to an electrophoresis power source. A constant voltage of 75-80 V was applied until the A40 (blue) dye began to exit the gel. Since the fractionator system used included an in-operation signal, after several seconds, the base of the fractionator illuminated, indicating that there was a complete electrical circuit. The expected run time may be ˜12 min to purify ≦40 nucleotide nucleic acids.

The blue dye in the loading buffer migrated with the 40 nucleotide nucleic acids. As it approached the lower gel surface, the blue dye band became very tight (the dye stacked).

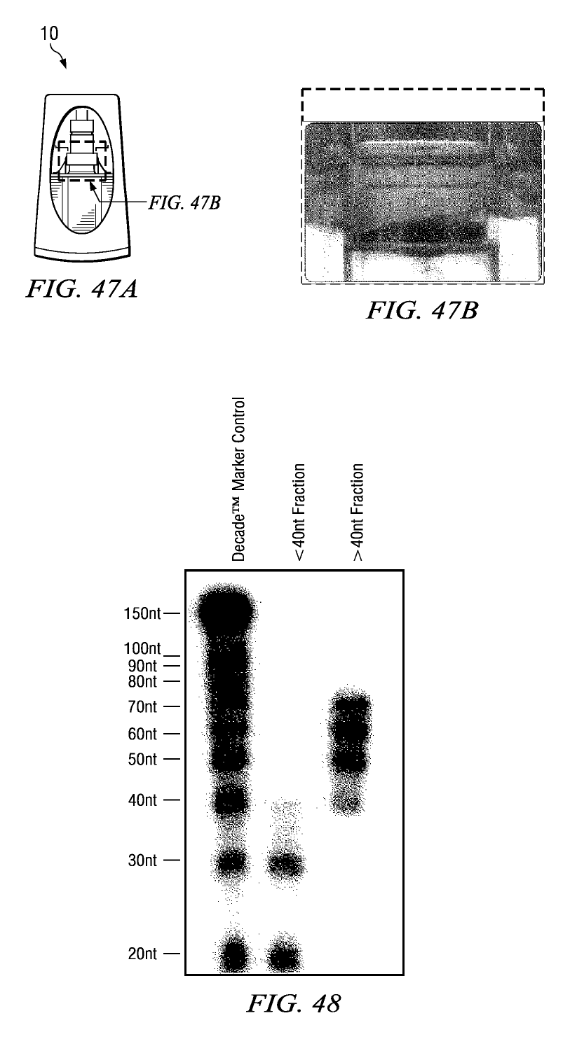

A nucleic acid fraction below about 40 nucleotides was collected by running the gel until the blue dye just began to migrate off the gel into the lower running buffer as shown in FIGS. 47A and 47B.

### Example 4

### Sample Recovery

A fractionator of the disclosure was opened to break the circuit before the electrophoresis power supply was shut off. After removing the pre-cast gel, the lower running buffer, which contains the <40 nucleotide nucleic acid fraction, was removed and placed on ice.

### Example 5

### Control Run

Decaderm Marker (Ambion, Cat. #7778) in a background of 10 μg mouse brain total RNA, was loaded onto a pre-cast fractionator gel cartridge and electrophoresed with a fractionator system of the disclosure. Two successive fractions were collected. For the first fraction, the lower buffer was collected and precipitated when the molecular weight marker had reached the lower end of the gel cartridge. After adding fresh lower buffer to the apparatus, the sample was electrophoresed for ten (10) more minutes. The second lower buffer fraction was then collected. Both fractions were precipitated and resolved by PAGE.

FIG. 48 shows the successful isolation of Ambion's Decade′″ Markers (RNA molecules between 10-150 nucleotides) using a fractionator system of the disclosure. In this example, two samples separated by the molecular weight marker were removed. Removed nucleic acid may be further purified (e.g., to remove small molecular contaminants) and/or concentrated using any technique including, without limitation, a glass fiber filter.

Extensive validation of a fractionator system of the disclosure confirms that greater than 95% of species longer than 40 nucleotides are excluded from the small RNA/DNA fraction when the run was terminated with a 40-base marker.

### Example 6

### Pre-Purification/Pre-Isolation

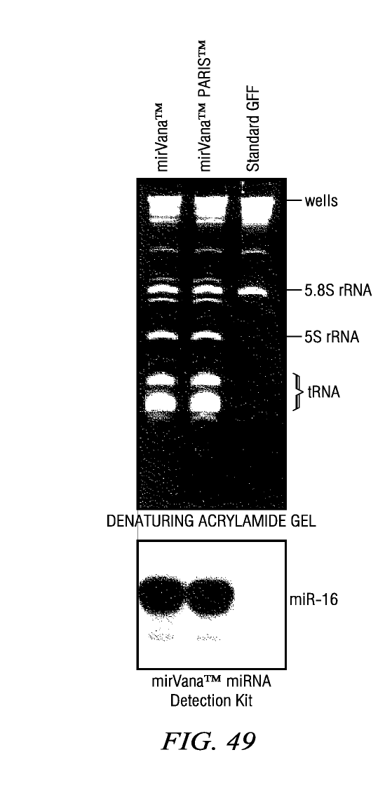

Total RNA was isolated from 1×10⁶ HeLa cells with the indicated Ambion kit as per protocol. Total RNA (1 μg) was resolved on a 15% denaturing acrylamide gel and stained with ethidium bromide or analyzed by solution hybridization assay with the mirVana™ miRNA Detection Kit (Ambion) and a probe specific for miR-16 prepared by in vitro transcription with the mirVana Probe Construction Kit (Ambion). The gel was exposed for 6 h at −80° C. Results are shown in FIG. 49.

As will be understood by those skilled in the art, other equivalent or alternative methods, devices, systems and compositions for separation and/or purification of a nucleic acid, a protein, and/or a carbohydrate according to embodiments of the present disclosure can be envisioned without departing from the essential characteristics thereof. For example, where a range is disclosed, the end points may be regarded as guides rather than strict limits. In addition, ranges disclosed herein are intended to include up to all possible subset ranges. For example, a range of from about 4% (v/v) polyacrylamide to about 8% (v/v) polyacrylamide constitutes a disclosure of, without limitation, from about 4% (v/v) polyacrylamide to about 5% (v/v), from about 4% (v/v) polyacrylamide to about 6% (v/v), from about 4% (v/v) polyacrylamide to about 7% (v/v), from about 4% (v/v) polyacrylamide to about 8% (v/v), from about 5% (v/v) polyacrylamide to about 6% (v/v), from about 5% (v/v) polyacrylamide to about 7% (v/v), from about 5% (v/v) polyacrylamide to about 8% (v/v), from about 6% (v/v) polyacrylamide to about 7% (v/v), from about 6% (v/v) polyacrylamide to about 8% (v/v), from about 7% (v/v) polyacrylamide to about 8% (v/v), and combinations thereof.

In some embodiments, methods, compositions, devices, and/or systems may be adapted to accommodate ergonomic interests, aesthetic interests, scale, or any other interests. Such modifications may influence other steps, structures and/or functions (e.g., positively, negatively, or insubstantially). A negative influence on function may include, for example, a loss of fractionation capacity and/or resolution. Yet, this loss may be deemed acceptable, for example, in view of ergonomic, aesthetic, scale, cost, or other factors.

In some embodiments, a device of the disclosure may be manufactured in either a handheld or a tabletop configuration, and may be operated sporadically, intermittently, and/or continuously. Individuals skilled in the art would recognize that additional separation methods may be incorporated, e.g., to partially or completely remove proteins, lipids, carbohydrates, and/or nucleic acids. Also, the temperature, pressure, and acceleration (e.g., spin columns) at which each step is performed may be varied.

All or part of a system of the disclosure may be configured to be disposable and/or reusable. From time to time, it may be desirable to clean, repair, and/or refurbish at least a portion of a device and/or system of the disclosure. For example, a reusable component may be cleaned to inactivate, remove, and/or destroy one or more contaminants (e.g., a nucleic acid and/or a nuclease). Individuals skilled in the art would recognize that a cleaned, repaired, and/or refurbished component is within the scope of the disclosure. In addition, individuals skilled in the art would recognize that a fractionator may further comprise an elution detector (e.g., an optical, spectrophotometric, fluorescence, and/or radioisotope detector) configured to monitor elution of a nucleic acid and/or marker of interest. Additionally, such detectors may function in a forward-scattering mode, a back-scattering mode, a reflection mode, and/or a transmission mode.

These equivalents and alternatives along with obvious changes and modifications are intended to be included within the scope of the present disclosure. Moreover, one of ordinary skill in the art will appreciate that no embodiment, use, and/or advantage is intended to universally control or exclude other embodiments, uses, and/or advantages. Expressions of certainty (e.g., “will,” “must,” and “can not”) may refer to one or a few example embodiments and not to all embodiments of the disclosure. Accordingly, the foregoing disclosure is intended to be illustrative, but not limiting, of the scope of the disclosure as illustrated by the following claims.

What is claimed is:

1. An apparatus for purifying a nucleic acid of interest from a sample within seconds to minutes, said system comprising:
an anode;
a collection chamber proximal to the anode and in electrical communication with the anode;
a sieving matrix in fluid and electrical communication with the collection chamber;
a loading chamber in fluid and electrical communication with the sieving matrix; and
a cathode in electrical communication with the loading chamber,
wherein the collection chamber is sized to contain or receive from about fifty (50) microliters to about eleven (11) milliliters,
wherein the loading chamber is sized to contain or receive from about fifty (50) microliters to about eleven (11) milliliters, and
wherein the sieving matrix comprises polyacrylamide at a concentration of from about 4% to about 20% (v/v) with an acrylamide:bisacrylamide ratio of from about 10:1 to about 100:1 and is from about one (1) millimeter to about twenty (20) millimeters in each dimension independently.

2. An apparatus for purifying a nucleic acid of interest according to claim 1, wherein the sieving matrix comprises polyacrylamide at a concentration of from about 8% to about 12% (v/v) with an acrylamide:bisacrylamide ratio of from about 10:1 to about 20:1.

3. An apparatus for purifying a nucleic acid of interest according to claim 2, wherein the sieving matrix comprises polyacrylamide at a concentration of from about 9% to about 11% (v/v) with an acrylamide:bisacrylamide ratio of from about 12:1 to about 16:1.

4. An apparatus for purifying a nucleic acid of interest according to claim 3, wherein the sieving matrix comprises polyacrylamide at a concentration of about 10% (v/v) with an acrylamide:bisacrylamide ratio of about 14:1.

5. An apparatus for purifying a nucleic acid of interest according to claim 1, wherein the sieving matrix has a generally cylindrical shape with a radius of from about one (1) millimeter to about five (5) millimeters and a length of from about five (5) millimeters to about twenty (20) millimeters.

6. An apparatus for purifying a nucleic acid of interest according to claim 5, wherein the radius is about of from about two (2) millimeter to about four (4) millimeters and a length of from about ten (10) millimeters to about fifteen (15) millimeters.

7. An apparatus for purifying a nucleic acid of interest according to claim 1, wherein the fluid communication between the loading chamber and the collection chamber is solely through the sieving matrix.

8. An apparatus for purifying a nucleic acid of interest according to claim 1, wherein the loading chamber further comprises a loading chamber buffer having tris(hydroxymethyl)aminomethane and boric acid at a molar ratio of from about 2:1 to about 1:1.

9. An apparatus for purifying a nucleic acid of interest according to claim 8, wherein the loading chamber buffer further comprises ethylene diamine tetra-acetic acid.

10. An apparatus for purifying a nucleic acid of interest according to claim 8, wherein the loading chamber buffer further comprises a non-ionic detergent.

11. An apparatus for purifying a nucleic acid of interest according to claim 10, wherein the non-ionic detergent comprises octylphenol ethoxylate.

12. An apparatus for purifying a nucleic acid of interest according to claim 8, wherein the loading chamber buffer has a pH above about 8.0.

13. An apparatus for purifying a nucleic acid of interest according to claim 8, wherein the loading chamber buffer has a pH below about 8.0.

14. An apparatus for purifying a nucleic acid of interest according to claim 1, wherein the collection chamber further comprises a collection chamber buffer having tris(hydroxymethyl)aminomethane and boric acid at a molar ratio of from about 2:1 to about 1:1.

15. An apparatus for purifying a nucleic acid of interest according to claim 1, wherein the loading chamber is sized to contain a volume of up to about two hundred (200) microliters.

16. An apparatus for purifying a nucleic acid of interest according to claim 1, wherein the loading chamber is sized to contain a volume of up to about four hundred (400) microliters.

17. An apparatus for purifying a nucleic acid of interest according to claim 1, wherein the loading chamber is sized to contain a volume of up to about one (1) milliliter.

18. An apparatus for purifying a nucleic acid of interest according to claim 1, wherein the collection chamber is sized to contain a volume of up to about two hundred (200) microliters.

19. An apparatus for purifying a nucleic acid of interest according to claim 1, wherein the collection chamber is sized to contain a volume of up to about four hundred (400) microliters.

20. An apparatus for purifying a nucleic acid of interest according to claim 1, wherein the collection chamber is sized to contain a volume of up to about one (1) milliliter.

21. An apparatus for purifying a nucleic acid of interest according to claim 1 further comprising a housing, wherein the housing encloses at least a portion of the collection chamber, the sieving matrix, the loading chamber, or combinations thereof.

22. An apparatus for purifying a nucleic acid of interest according to claim 21, wherein the collection chamber releasably contacts at least a portion of the housing.

23. An apparatus for purifying a nucleic acid of interest according to claim 1 further comprising a sieving matrix wall, wherein at least a portion of the sieving matrix contacts at least a portion of the sieving matrix wall.

24. An apparatus for purifying a nucleic acid of interest according to claim 23, wherein the sieving matrix wall releasably contacts the collection chamber.

25. An apparatus for purifying a nucleic acid of interest according to claim 1 further comprising a plurality of collection chambers, sieving matrices, loading chambers, or combinations thereof.

26. An apparatus for purifying a nucleic acid of interest according to claim 1 further comprising a safety cut-off switch configured to conditionally block or interrupt electrical communication between the anode and cathode.

27. An apparatus for purifying a nucleic acid of interest according to claim 1 further comprising a power source in electrical communication with the anode, the cathode, or both the anode and the cathode.

28. A system for purifying a nucleic acid of interest from a sample within seconds to minutes, said system comprising:
an anode;
a collection chamber proximal to the anode and in electrical communication with the anode;
a sieving matrix in fluid and electrical communication with the collection chamber;
a loading chamber in fluid and electrical communication with the sieving matrix;
a cathode in electrical communication with the loading chamber;
a housing enclosing at least a portion of the collection chamber, a sieving matrix, and a loading chamber;
a power source in electrical communication with the anode, the cathode, or both the anode and the cathode; and
a safety cut-off switch configured to conditionally block or interrupt electrical communication between the anode and cathode,
wherein the collection chamber is sized to contain or receive from about fifty (50) microliters to about two (2) milliliters,
wherein the loading chamber is sized to contain or receive from about fifty (50) microliters to about two (2) milliliters, and
wherein the sieving matrix has a generally cylindrical shape with a radius of from about two (2) millimeters to about five (5) millimeters and a length of from about eight (8) millimeters to about sixteen (16) millimeters and comprises polyacrylamide at a concentration of from about 8% to about 12% (v/v) with an acrylamide:bisacrylamide ratio of from about 10:1 to about 20:1.

29. A system for purifying a nucleic acid of interest according to claim 28 further comprising
a second collection chamber in electrical communication with the anode;
a second sieving matrix in fluid and electrical communication with the second collection chamber; and
a second loading chamber in fluid and electrical communication with the second sieving matrix,
wherein the second collection chamber is sized to contain or receive from about fifty (50) microliters to about two (2) milliliters,
wherein the second loading chamber is sized to contain or receive from about fifty (50) microliters to about two (2) milliliters, and
wherein the second sieving matrix has a generally cylindrical shape with a radius of from about two (2) millimeters to about five (5) millimeters and a length of from about eight (8) millimeters to about sixteen (16) millimeters and comprises polyacrylamide at a concentration of from about 8% to about 12% (v/v) with an acrylamide:bisacrylamide ratio of from about 10:1 to about 20:1.

30. A system for purifying a nucleic acid of interest according to claim 29, wherein the collection chamber and the second collection chamber are sized to contain different volumes.

31. A system for purifying a nucleic acid of interest according to claim 29, wherein the collection chamber and the second collection chamber are sized to contain substantially the same volume.

32. A system for purifying a nucleic acid of interest according to claim 29, wherein the sieving matrix and the second sieving matrix independently have different sizes, shapes, and compositions.

33. A system for purifying a nucleic acid of interest according to claim 29, wherein the sieving matrix and the second sieving matrix have substantially the same size, shape, and composition.

34. A system for purifying a nucleic acid of interest according to claim 29, wherein the loading chamber and the second collection chamber are sized to contain substantially the same volume.

35. A system for purifying a nucleic acid of interest according to claim 29, wherein the loading chamber and the second collection chamber are sized to contain different volumes.

36. A disposable sieving matrix cartridge for purifying a nucleic acid of interest from a sample within seconds to minutes, said disposable sieving matrix cartridge comprising:
a sieving matrix having a generally cylindrical shape with a radius of from about two (2) millimeters to about five (5) millimeters and a length of from about eight (8) millimeters to about sixteen (16) millimeters and comprising polyacrylamide at a concentration of from 8% to about 12% (v/v) with an acrylamide:bisacrylamide ratio of from about 10:1 to about 20:1; and
a sieving matrix wall surrounding the sieving matrix and defining an upper chamber,
wherein at least a portion of the sieving matrix contacts at least a portion of the sieving matrix wall.

37. A system for purifying a nucleic acid of interest within seconds to minutes, said system comprising:
a fractionator having a housing, a first electrode, a second electrode spaced away from the first electrode, and a collection chamber proximal to the second electrode; and
a pre-cast sieving matrix cartridge having an upper buffer chamber and an elongate polyacrylamide gel,
wherein (1) the upper buffer chamber is in fluid communication with one end of the polyacrylamide gel, (2) the polyacrylamide gel comprises bisacrylamide and from about 4% to about 20% (v/v) acrylamide with an acrylamide:bisacrylamide ratio of from about 10:1 to about 100:1, (3) the lower buffer chamber is sized to contain or receive from about fifty (50) microliters to about eleven (11) milliliters and is in fluid communication with the other end of the elongate polyacrylamide gel, (4) the first electrode is in electrical communication with the upper buffer chamber, and (5) the second electrode is in electrical communication with the collection chamber.

38. A method for purifying a compound of interest within seconds to minutes, said method comprising:
(a) providing a fractionator having a housing, a first electrode, a second electrode spaced away from the first electrode, and a lower buffer chamber proximal to the second electrode;
(b) providing a pre-case sieving matrix cartridge having a loading chamber and an elongate sieving matrix,
(c) contacting a collection chamber buffer with the collection chamber wherein the collection chamber buffer is contained within at least a portion of the collection chamber;
(d) contacting at least a portion of the collection chamber buffer with at least a portion of the sieving matrix, wherein the sieving matrix and the collection chamber are in fluid communication;
(e) contacting at least a portion of the sieving matrix with the loading chamber wherein the sieving matrix and the loading chamber are in fluid communication;
(f) contacting a loading chamber buffer with the loading chamber wherein the loading chamber buffer is contained within at least a portion of the loading chamber;
(g) contacting at least a portion of the loading chamber buffer with a sample having the compound of interest and at least one other compound;
(h) contacting at least a portion of the sieving matrix with at least a portion of the sample under conditions that permit differential sieving of the compound of interest and the at least one other compound; and
(i) receiving the compound of interest in at least a portion of the receiving buffer to the substantial exclusion of the at least one other compound, wherein the compound of interest is thereby purified from the at least one other compound,
wherein the compound of interest is selected from the group consisting of a carbohydrate, a protein, and a nucleic acid,
wherein the sieving matrix has a generally cylindrical shape with a radius of from about two (2) millimeters to about five (5) millimeters and a length of from about eight (8) millimeters to about sixteen (16) millimeters and comprises polyacrylamide at a concentration of from about 8% to about 12% (v/v) with an acrylamide:bisacrylamide ratio of from about 10:1 to about 20:1, and
wherein the time from the contacting at least a portion of the loading chamber buffer with a sample having the compound of interest and at least one other compound to the receiving the compound of interest in at least a portion of the receiving buffer to the substantial exclusion of the at least one other compound is less than about fifteen (15) minutes.

39. A method for purifying a compound of interest according to claim 38, wherein the time from the contacting at least a portion of the loading chamber buffer with a sample having the compound of interest and at least one other compound to the receiving the compound of interest in at least a portion of the receiving buffer to the substantial exclusion of the at least one other compound is less than about twelve (12) minutes.

40. A method for purifying a compound of interest according to claim 39, wherein the time from the contacting at least a portion of the loading chamber buffer with a sample having the compound of interest and at least one other compound to the receiving the compound of interest in at least a portion of the receiving buffer to the substantial exclusion of the at least one other compound is less than about ten (10) minutes.
